(19) United States
(12) Patent Application Publication
(10) Pub. No.: US 2007/0270926 A1
(43) Pub. Date: Nov. 22, 2007

(54) Device for Cooling a Body

(75) Inventor: Jose Antonio Serrano Molina, Montcada I. Reixac (ES)

Correspondence Address: WOLF, BLOCK, SHORR AND SOLIS-COHEN LLP, 250 PARK AVENUE, 10TH FLOOR, NEW YORK, NY, 10177, US

(73) Assignee: Prendas Capricornio, S.L, Sant Cebria de Viialta (ES)

(21) Appl. No.: 11/569,187

(22) PCT Filed: May 13, 2005
(86) PCT No.: PCT/IB05/01448

§ 371 Date: Nov. 16, 2006

(30) Foreign Application Priority Data
May 19, 2004 (ES) P200401195

(51) Int. Cl. A61F 7/00

(52) U.S. Cl. 607/108; 2/69

(57) ABSTRACT

Device (1) for cooling a body, which comprises a garment (2) in contact with said body, a coolant for cooling said garment (2) and means of storing said coolant, said garment (2) including a layer of conductive fabric with a thermal conductivity suitable for transmitting by contact the heat to said coolant stored in said storage means and is characterised in that said coolant is a cryogenic fluid and in that said cryogenic fluid is liquid nitrogen (4). The device possesses high coolant power, since it permits the use of coolant substances at very low temperature, adapts easily to the body on which it is applied and is very safe.

This invention relates to a device for cooling a body, particularly the human body, of the type that comprises a garment in contact with said body and a coolant for cooling said garment.

### BACKGROUND OF THE INVENTION

Known in the art are devices for cooling the human body that include a clothing garment in contact with said body and a network of tubes incorporated into the garment and through which there circulates the coolant or substance that absorbs a large amount of heat.

U.S. Pat. No. 5,438,707 discloses a device for cooling that comprises a garment with a network of small-dimension tubes that include a plurality of small openings. The cooling system is based on feeding into said tube network a pressurised gas, preferably air or carbon dioxide, and on the rapid expansion of said compressed gas through the small openings in said tubes. Upon expanding, the gas cools and with it also the air in the vicinity of the person's body. The cooling effect caused by expansion of the gas is complemented by the flow of air and gases generated with said expansion, which ventilates and refreshes the wearer's body.

U.S. Pat. No. 4,738,119, discloses a device for cooling that also comprises a garment with a network of tubes which, in this case, have a structure of micro-pores. The cooling system is based on high-pressure injection into said tubes of liquid carbon dioxide, which expands and is converted into a mixture of solid carbon dioxide (dry ice) and gaseous carbon dioxide. The solid carbon dioxide sublimates to carbon dioxide gas, and is released through the micro-pores in the tubes into the compartments formed between the two fabrics that make up the garment, in which said tubes are housed. From said compartments the gas flows towards the user's body through the inner fabric which is permeable. While the sublimation takes place, the solid carbon dioxide absorbs a large amount of heat, producing a cooling effect on the garment wearer.

The devices disclosed in the aforesaid patents have the disadvantage that the cooling of the garment and of its user depends upon incorporating into said garment a network of tubes through which the coolant substance circulates. This involves a number of disadvantages, for example that the garment is rather inflexible and that any breakage in said garment that affects said network of tubes renders the cooling device ineffective. Moreover, it should be taken into account that breakage of said network means leakage of said coolant and, in function of the type of substance employed, could involve a serious hazard for the user of the device.

Another disadvantage of the devices disclosed in the North American patents is that they are not suitable for using with very-low-temperature coolants, since in said devices the coolant comes into contact with the garment user.

### DESCRIPTION OF THE INVENTION

The object of this invention is to resolve the disadvantages mentioned by developing a device for cooling a body that presents the advantages that are set out below.

In accordance with this objective, the device of the present invention comprises a garment in contact with said body, a coolant for cooling said garment and means of storing said coolant, and is characterised in that said garment includes a layer of conductive fabric with a thermal conductivity suitable for transmitting by contact the heat to said coolant stored in said storage means.

Thanks to the fact that the heat taken up by the layer of conductive fabric is transmitted by contact to the coolant stored in said storage means, the device has the advantages that the coolant never comes into direct contact with the body onto which the device is applied and that it is not necessary to incorporate a network of tubes into said garment, since the coolant remains stored at all times in said storage means. Any eventual breakage of the garment therefore does not put the device out of service, while said garment is flexible and adapts easily to the body.

Another advantage presented by the device of the invention is that, as there is no direct contact between the coolant and the body, it is possible to use as coolant substances at very low temperatures without said body being negatively affected by such low temperatures. Thanks to this, the coolant power of the device of the invention can be very high, thus making it suitable for bodies subjected to environments with very extreme temperatures, such as the human bodies of professionals such as firefighting personnel, boiler operatives, etc.

In the present invention, coolant is taken to mean any substance which, when it undergoes a change of phase, absorbs a large amount of latent heat in relation to its volume, thus producing a cooling effect, or any substance capable of absorbing a large amount of heat without changing phase.

Thermal conductivity is taken to mean a property of a material that indicates the speed at which heat is transferred within said material.

Preferably, said thermal conductivity of the conductive fabric exceeds 50 W/m.k.

Preferably, said device includes means for transmitting by contact the heat coming from said layer of conductive fabric to said coolant stored in said storage means.

The existence of said transmitting means avoids direct contact of the layer of conductive fabric with said coolant or said storage means that contain said coolant. It is thereby possible to provide for regulation of the transmission of heat of the device and, therefore, regulation of the coolant effect.

Advantageously, said means for transmitting heat include at least one metallic piece in contact with said layer of conductive fabric. Said material presents the advantage of possessing high thermal conductivity.

Also advantageously, said at least one metallic piece comprises a plate for contact with said layer of conductive fabric and a metallic bar provided between said plate and the coolant.

Said plate increases the contact surface area in which heat exchange takes place between the conductive fabric and the coolant. The total heat transmitted is thus increased, thereby achieving a rapid cooling effect.

The metallic bar provided between the plate and the coolant constitutes an element that can be acted upon in order to regulate the total heat transmitted by the contact plate.

Preferably, said device includes means to regulate transmission of the heat to said coolant stored in said storage means. Thanks to these characteristics, it is possible to control the heat transmitted by the device, adjusting it to the needs of the body on which said device is applied.

Also preferably, said coolant is a cryogenic fluid.

Advantageously, said cryogenic fluid is liquid nitrogen. Said fluid has the advantage that it can absorb a large amount of heat, since it is at a temperature of −196° C. It is moreover non-toxic, non-flammable and odourless, while being also of low cost.

Preferably, when the coolant employed is at very low temperatures, said garment includes at least one layer of fabric placed between said layer of conductive fabric and said body, in order to prevent the temperature of the body on which the device is applied falling below a pre-established value.

Preferably, said storage means include at least one tank.

Also preferably, said garment includes a layer of strong fabric over the layer of conductive fabric in order to facilitate the attachment of said tank.

Advantageously, when said coolant is a cryogenic fluid, said tank includes means for releasing said coolant in gaseous phase.

### BRIEF DESCRIPTION OF THE DRAWINGS

For a better understanding of all that has been set out, some drawings are attached that show, schematically and solely by way of non-restrictive example, a practical case of embodiment.

In said drawings,

FIG. 1 is a perspective view of a preferred embodiment of a device of the invention fitted on a human body and including a plurality of coolant tanks.

FIG. 2 shows a section of one of the tanks and metallic piece of FIG. 1.

FIG. 3 shows a detail of the section of one of the tanks and metallic piece of the device of FIG. 1, in deactivated position.

FIG. 4 shows a detail of the section of one of the tanks and metallic piece of the device of FIG. 1, in activated position.

### DESCRIPTION OF A PREFERRED EMBODIMENT

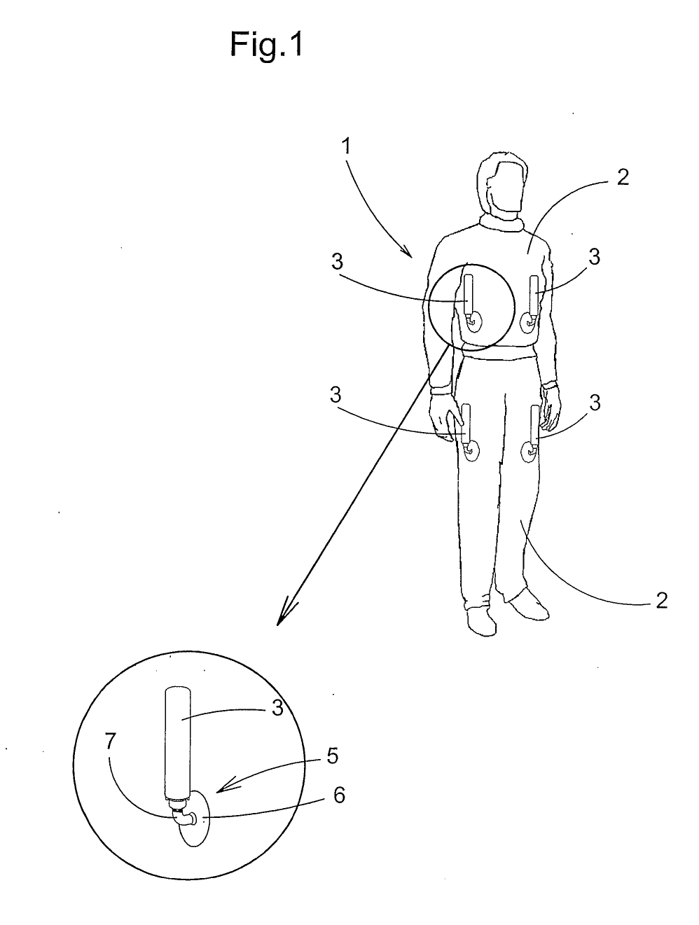

FIG. 1 shows a preferred embodiment of a device 1 of the invention fitted on the human body of any user, which includes a garment 2 and a plurality of tanks 3 with liquid nitrogen 4, arranged on said garment 2. The garment 2 includes a layer of conductive fabric, not shown, which possesses thermal conductivity suitable for transmitting by contact the heat to the liquid nitrogen 4 stored in said tanks 3.

Each one of said tanks 3 includes a metallic piece 5, in contact with said layer of conductive fabric, that transmits the heat to the coolant stored in the tank 3. Said metallic piece 5 includes a plate 6 in contact with the conductive fabric and an elbowed solid bar 7, provided between said plate 6 and said layer of conductive fabric.

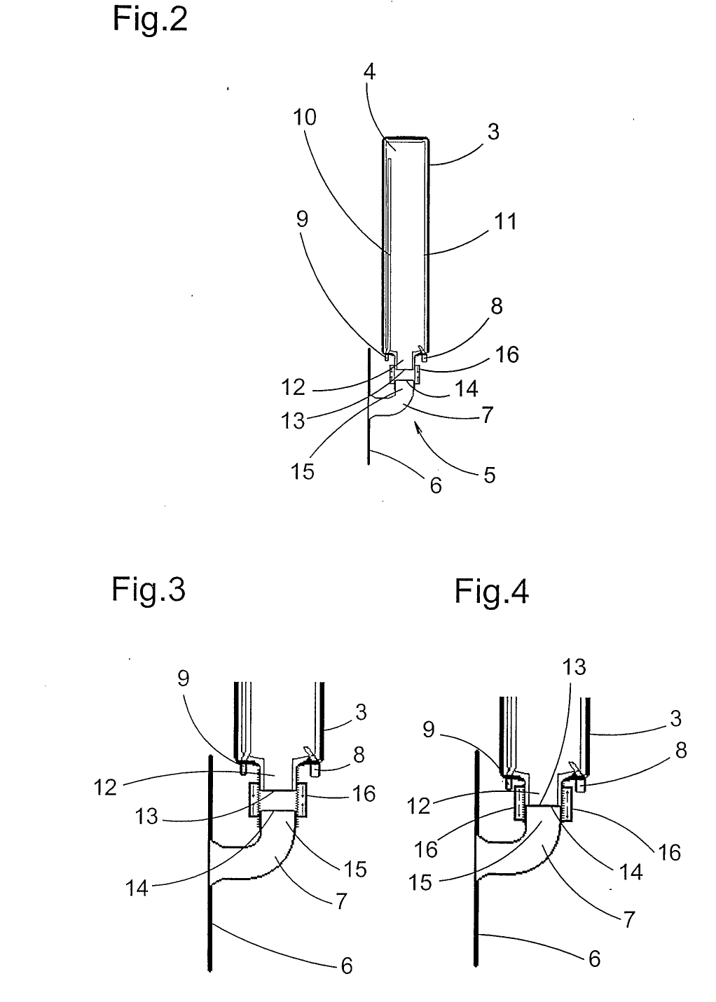

As can be seen in FIG. 2 that shows a section of one of the tanks 3 and metallic piece 5 of FIG. 1, the lower part of the tank 3 includes a valve 8 through which the liquid nitrogen 4 is introduced, and another valve 9 through which the nitrogen 4 is released once converted into gas. The nitrogen 4 in gaseous phase accumulates in the upper part of the tank 3, and so a tube 10 is provided to take it to the valve 9 situated in the lower part of said tank 3.

In order to keep the nitrogen 4 in liquid phase, the tank 3 is provided with thermal insulation means that consist of two layers 11 between which a vacuum has been generated.

As FIG. 2 also shows, the tank 3 includes a prolongation 12 whose free end forms a flat zone 13 without thermal insulation. Said flat zone 13 is specially designed to transmit by contact the heat coming from the layer of conductive fabric to the liquid nitrogen 4 inside the tank 3. The transmission by contact is carried out through the flat side 14 of the end 15 of the solid bar 7 of the metallic piece 5.

In order to regulate the transmission of heat to the coolant, in this case liquid nitrogen 4, the device 1 includes a threaded piece 16 mounted on the external walls of the tank 3 prolongation 12 and the end 15 of the solid bar 7. When said threaded piece 16 is actuated in one direction or another the prolongation 12 and the end 15 move closer towards or further away from each other, thereby enabling the heat transmission to be regulated, from an activated position to a deactivated position of the device 1.

FIGS. 3 and 4 show a detail of one of the tanks 3 and metallic piece 5 of device 1, in deactivated and activated positions, respectively.

As can be observed in FIG. 4, in the activated position of the device 1, the flat zone 13 of the prolongation 12 of the tank 3 is in contact with the flat side 14 of the end 15 of the solid bar 7. The heat from the layer of conductive fabric of the garment 2 is thus transmitted to the liquid nitrogen 4 through the plate 6, the bar 7 and the flat zone 13 of the prolongation 12 of the tank 3.

In the deactivated position of the device 1, as shown in FIG. 3, the flat side 14 of the bar 7 and the flat zone 13 of the prolongation 12 of the tank 3, are spaced apart, so that the transmission of heat to the coolant is negligible.

There follows a description of the mode of operation of the device 1 of the present preferred embodiment of the invention.

Firstly, coolant must be loaded into each of the tanks 3 of the garment 2. For this purpose, it must be ensured that said device 1 is in deactivated position.

The coolant, in this case liquid nitrogen 4, is fed into the tank 3 through the valve 8. The filling is carried out from a tank fitted with a pressurised extraction device, using a hose that connects said tank with the tank 3 of the device 1. The air or gaseous nitrogen displaced inside the tank 3 by the liquid nitrogen 4 that is loaded is released to the outside through the valve 9.

Once coolant 4 has been loaded, the user dons the device 1, so that the garment 2 is left in direct contact with the skin. Any other garment can be worn over said device 1, such as a fire-extinction suit where the user is a firefighter.

The device 1 must be activated a few minutes before the moment the coolant effect is to be achieved. In order to activate it, the piece 16 is moved so that the flat zone 13 of the tank 3 prolongation 12 comes into contact with the flat side 14 of the solid bar 7. Said solid bar 7 is connected to the plate 6, which is in turn connected to the layer of conductive fabric of the garment 2. Said layer of conductive fabric absorbs the heat received by the user's body, which heat can come from internal sources (physical exercise) or from external sources (fire, in the case of firefighters).

Once the device 1 has been activated, the heat from the layer of conductive fabric is transmitted by contact to the liquid nitrogen 4 stored inside the tank 3.

The liquid nitrogen 4 absorbs a large amount of heat when it changes phase and becomes a gas at −196° C., for which reason it possesses considerable coolant power that makes it highly suitable for use as coolant for the device 1 when the body to which said device 1 is applied is subjected to high-temperature environments. This is the case, for example, of the human bodies of firefighters.

The liquid nitrogen 4 converted to gas accumulates in the tank 3 until it reaches a pressure and temperature suitable for releasing it through the valve 9. The release of this gas provides the garment 2 with extra cooling, for when it is released it is still at a temperature lower than that of said garment.

The coolant effect of the device 1 is still maintained after all the liquid nitrogen 4 has been converted to gas, since the change of phase of the nitrogen occurs at a temperature of −196° C. However, as this gas warms up and is released through the valve 9, the coolant effect of the device 1 reduces until the layer of conductive fabric begins to be unable to absorb the heat it receives. At this point the user, when in an extreme-temperature environment, must proceed to evacuate the premises or to replace the device 1 used with another that has been recently loaded with coolant.

Surprisingly, the device 1 of the present invention possesses high coolant power, since it permits the use of coolant substances at very low temperature, adapts easily to the body to which it is applied, and is very safe, for the coolant effect is achieved without need for the coolant 4 to come into direct contact with said body.

Although a preferred embodiment of the invention has been described, in which the body to which the device 1 is applied is a human body, it should be borne in mind that said device 1 can be applied to cool any kind of bodies, whether or not they be living bodies, such as foods and drinks.

1. Device (1) for cooling a body, comprising a garment (2) in contact with said body, a coolant for cooling said garment (2) and storage means of said coolant, wherein said garment (2) includes a layer of conductive fabric with a thermal conductivity suitable for transmitting by contact the heat to said coolant stored in said storage means, wherein said coolant is a cryogenic fluid and wherein said cryogenic fluid is liquid nitrogen (4).

2. Device for cooling a body according to claim 1, further comprising means for transmitting by contact coming from said layer of conductive fabric to said coolant stored in said storage means.

3. Device for cooling a body according to claim 2, wherein said means for transmitting heat include at least one metallic piece (5) in contact with said layer of conductive fabric.

4. Device for cooling a body according to claim 3, wherein said metallic piece (5) comprises a plate (6) for contact with said layer of conductive fabric and a metallic bar (7) provided between said plate (6) and the coolant.

5. Device for cooling a body according to claim 1, further comprising means for regulated transmission of the heat to said coolant stored in said storage means.

6. Device for cooling a body according to claim 1, wherein said garment (2) includes at least one layer of fabric placed between said layer of conductive fabric and said body, in order to prevent the temperature of the body on which the device (1) is applied falling below a pre-established value.

7. Device for cooling a body according to claim 1, wherein said storage means include at least one tank (3).

8. Device for cooling a body according to claim 7, wherein said tank includes means (9) for releasing said coolant in gaseous phase.
